(19) United States
(12) Patent Application Publication
(10) Pub. No.: US 2009/0088702 A1
(43) Pub. Date: Apr. 2, 2009

(54) METHODS FOR MANUALLY INJECTING/ASPIRATING FLUIDS THROUGH SMALL DIAMETER CATHETERS AND NEEDLES AND MANUAL INJECTION/ASPIRATION SYSTEMS INCLUDING SMALL DIAMETER CATHETERS AND NEEDLES

(76) Inventor: Shawn P. Fojtik, Park City, UT (US)

Correspondence Address: Mallinckrodt Inc., 675 McDonnell Boulevard, HAZELWOOD, MO, 63042, US

(21) Appl. No.: 11/865,626

(22) Filed: Oct. 1, 2007

(51) Int. Cl. A61M 5/178

(52) U.S. Cl. 604/223

(57) ABSTRACT

A system for increasing rates at which fluids may be manually forced through injection/aspiration elements, such as catheters and needles, includes the injection/aspiration elements, as well as a syringe including a pair of crossed handles, one associated with the barrel of the syringe, the other associated with the plunger of the syringe, to provide a mechanical advantage. The system may include and injection/aspiration element of small (e.g., 0.052 inch or smaller) inner diameter. Such systems enable the use of a single hand to hold and operate a syringe in a variety or procedures, including angiography, angioplasty, discography, glue/cement injection, and a variety of aspiration procedures (e.g., biopsy, sampling, media removal, etc.).

### FIELD OF INVENTION

The present invention relates generally to methods that employ hand-held, manually operated syringes with crossing handles and catheters for introducing fluids into the body of a subject and/or for withdrawing fluids from the body of the subject by squeezing (i.e., bringing together) or opening (i.e., moving apart) the syringe handles and, more specifically, to fluid introduction and/or withdrawal methods that employ catheters having sizes of about five French or less. The present invention also relates to systems including hand-held syringes and catheters having sizes of about five French or less.

### BACKGROUND OF RELATED ART

Systems including catheters and syringes are used for a variety of medical procedures in which fluids are introduced or injected into and/or aspirated or withdrawn from the body of the subject. Examples of these types of medical procedures include a number of angiography, angioplasty, and discography techniques, to name only a few.

Angiography is a procedure by which vessels (e.g., arteries, veins, etc.) or cavities (e.g., heart chambers, etc.) within a subject's body may be visualized. Angiography procedures may be used to evaluate vessels or cavities in a number of locations throughout the body, including coronary blood vessels, cerebral blood vessels, and retinal blood vessels. Angiography typically includes the introduction of a radio contrast agent, which is also commonly referred to as a “contrast agent,” “contrast media,” “contrast solution,” or even more simply as “contrast,” into each vessel or cavity that is to be pictured. The use of contrasting media is usually necessary since it is often difficult to distinguish between tissues and any openings or cavities defined by the tissues when x-ray and CAT scan technologies are employed. In the cases of blood vessels and heart chambers, blood is also difficult to distinguish from the tissues through which the blood is carried. While the use of contrasting media in angiography procedures is widespread, it is also somewhat undesirable due to the act that it involves the introduction of a foreign substance into the body.

Conventionally, hand-held syringes have been used in connection with relatively large diameter catheters in angiography and other procedures. Typically, basic syringes, which include a barrel with flanges configured for engagement by a user's fingers and a plunger with an end configured to receive and be moved by the user's thumb, or control syringes, which are similar to basic syringes, but include loops for receiving a user's fingers and thumb have been used to provide the force needed to inject the contrast through a catheter and to a desired location of a subject's body. Since basic syringes and control syringes are primarily thumb operated, and the combination of two fingers and a thumb usually does not provide a great deal of force, catheters with relatively large openings (i.e., catheters having sizes (outer dimensions) of 6 French (F) and larger) are typically used to reduce the amount of resistance on contrast flowing through the catheter and, thus, the amount of force that must be applied by the syringe user to cause the contrast to flow through the catheter. Moreover, when operated by hand, conventional hand-held, due to variations in thumb positioning relative to the fingers during use due to the requirement that the thumb move toward the fingers (in injection) or away from the fingers (in aspiration), hand-operated syringes typically do not deliver or receive fluids at substantially constant rates over the entire course of moving the plunger through the barrel. Rather, the rate of injection or aspiration decreases significantly before movement of the plunger into or out of the barrel is complete.

Research has shown that when conventional hand-held, hand-operated syringes are used, 6F or larger catheters are required to ensure that contrast is introduced into the blood vessels of a subject at a quick enough rate (i.e., 5 ml/sec or faster) to ensure dense, uniform opacification of blood vessels with contrast (Gardiner, GA, et al., “Selective Coronary Angiography Using A Power Injector,” Am. J. Roentgenol., 146(4):831-33 (1986)), while reducing contrast wastage (Ganeshkumar, A, et al., “Traditional Versus Automated Injection Contrast System in Diagnostic and Percutaneous Coronary Interventional Procedures: Comparison of the Contrast Volume Delivered,” J. Invasive Cardiol., 16(7):360-62 (2004)) and contrast-induced nephropathy (i.e., kidney damage) (Call, J, et al., “Automated Contrast Injection in Contemporary Practice during Cardiac Catheterization and PCI: Effects on Contrast-Induced Nephropathy,” J. Invasive Cardiol., 18(10):469-71 (2006)). Nonetheless, due to their relatively large size (i.e., 6F and larger), these catheters are known to cause discomfort and arterial complications (Saito, T, et al., “Evaluation of New 4 French Catheters by Comparison to 6 French Coronary Artery Images,” J. Invasive Cardiol., 11(1):13-30 (1999) (hereinafter “Saito”).

Despite the advantages of smaller (than 6 F) catheters, their small lumens increase resistance to flow, diminishing the rates at which contrast may be introduced to a desired location to unacceptably low levels. Consequently, power injectors have been required to deliver contrast with catheters that are smaller than 6F.

Angioplasty (or percutaneous transluminal angioplasty (PTA)) is a technique by which the lumens through blood vessels are mechanically widened. When the manipulated blood vessels are associated with the heart, the procedure is referred to as “coronary angioplasty.” “Peripheral angioplasty” includes angioplasty procedures that are effected on non-coronary blood vessels, such as renal arteries, the carotid arteries, and blood vessels in legs. Angioplasty procedures require catheters that often include angioplasty balloons at or near the distal ends thereof. Angioplasty balloons inflate, or expand or dilate, and deflate under control of a so-called “angioplasty inflator” (e.g., by introducing air or other gases into the catheter). Sometimes, the catheters and/or angioplasty balloons are used to position stents, which hold blood vessels open, at desired locations within the blood vessels. Catheter-introduced drug therapies may also be performed in connection with angioplasty.

The angioplasty inflators that are typically used during angioplasty processes are relatively complex syringes that include pressure gauges and plunger locks. As they are often used in connection with angioplasty balloons, angioplasty inflators are typically configured to deliver the relatively high pressures that are required to inflate the angioplasty balloons. Use of an angioplasty inflator requires at least two hands for simple tasks such as locking the plunger in place relative to the barrel, as well as for microadjustment of the location of the plunger along the length of the barrel. Thus, when these tasks are performed, a single user cannot hold the catheter in place to prevent its ejection from the body of a subject as pressurized air is introduced into the catheter.

In discography, sailine, contrast media, or a combination thereof is introduced into an intervertebral disk, which is located between two vertebrae of the spinal column of a subject. Typically, the skin and muscle of the subject is pierced by a first, guide needle, which is inserted until it reaches the outer surface of the disk. A smaller, second needle, is then inserted into the first needle, and into the disk, near its center. Contrast media is then (typically manually) injected into the disk through the second needle. Leakage of the contrast media from the center of the disk, which may be visualized by x-ray or CAT scan, is indicative of damage that may be the cause of back pain. To facilitate the manual injection of contrast media into a disk at a suitable rate, the contrast-injection needles that are used are relatively large (e.g., about 16 gauge (i.e., with an outer diameter of about 1/16 inch (about 1.59 mm) and an inner diameter of about 0.047 inch (about 1.2 mm)). Even larger guide needles (typically about 13 gauge (i.e., having an outer diameter of about 1/13 inch (about 1.95 mm)) and an inner diameter of about 0.071 inch (about 1.8 mm) or larger than 13 gauge) are needed to accommodate such large contrast-injection needles. As a consequence of the use of large needles, subjects that undergo discography usually experience significant post-procedure pain. Sometimes tissue damage, including damage to an evaluated disk, may also occur. Due to these undesirable aspects of discography, it is a procedure that is typically reserved for subjects that suffer from chronic back pain, and is not even used by some spine care physicians.

Syringes, including both manually operated and automated syringes, of ever-increasing complexity have been developed to facilitate the introduction and/or withdrawal of fluids at high pressure. Unfortunately, the mechanisms that are intended to enable fluid introduction at high pressures or rates also eliminate any of the tactile feedback that is often highly valued by physicians since it allows them to instinctively and immediately respond to complications or other unforeseen events that might occur during a particular procedure. Furthermore, these complex devices, particularly power syringes, are very costly to use, in terms of both money and time.

Accordingly, there are needs for processes, apparatus, and systems by which fluids may be manually introduced into or withdrawn from the body of a subject at a desirably high rate and/or under a desirably high pressure using minimally invasive apparatus while providing tactile feedback and requiring one user.

### SUMMARY

The present invention includes methods for introducing fluids into and/or withdrawing fluids from the body of a subject. Such a method includes use of a hand-held, manually operated syringe with crossing handles and a catheter that has a size of about five French or less, or another “small inner diameter element,” such as a needle, with an inner diameter that is the same as or less than that of a five French catheter. In one embodiment of such a method, fluids are introduced into the body or withdrawn from the body through a small inner diameter element at a rate of about 5 ml or more, even 10 ml or more, per second. By enabling the use of smaller elements (e.g., catheters, needles, etc.) in invasive processes, systems that incorporate teachings of the present invention facilitate access to sites that were not previously accessible in systems that included manually operated syringes where with larger catheters or needles were required to provide desired delivery or aspiration rates, reduce the potential for procedure-related complications, reduce discomfort to subjects, and/or allow subjects to heal faster once a procedure is complete. In another embodiment, fluids are introduced into the body or withdrawn from the body through a catheter, needle, or the like at a pressure and rate that exceeds that available with conventional hand-held syringes. In still another embodiment, a hand-held manually operated syringe with crossing handles may be used to cause fluids to move through a catheter with a lumen that is partially occupied by another element, such as a wire or the like.

Another aspect of the present invention includes systems for introducing and/or aspirating fluids from the body of a subject. An embodiment of such a system includes a hand-held, manually operated syringe with handles that are arranged to provide a user with a mechanical advantage without eliminating tactile feedback, and a catheter with a size of about five French or less.

Other features and advantages of the present invention will become apparent to those of ordinary skill in the art through consideration of the ensuing description, the accompanying drawings, and the appended claims.

### BRIEF DESCRIPTION OF THE DRAWINGS

In the drawings, which depict features of various examples of embodiments of the present invention:

FIG. 1 is a schematic representation of a system that includes a hand-held, hand-operated syringe and a catheter having a size of less about five French or less and configured, for example, for use in angiography processes;

FIG. 1A schematically shows use of an injection syringe in the system of FIG. 1;

FIG. 1B schematically shows use of an aspiration syringe in the system of FIG. 1;

FIG. 2 is a schematic representation of a system including a hand-held syringe and a needle, with the syringe configured to be operated, for example, in discography procedures, with one hand; and

FIG. 3 is a schematic representation of a system that includes a hand-held, hand-operated syringe and an angioplasty catheter, which may, of course, be used in angioplasty procedures.

### DETAILED DESCRIPTION

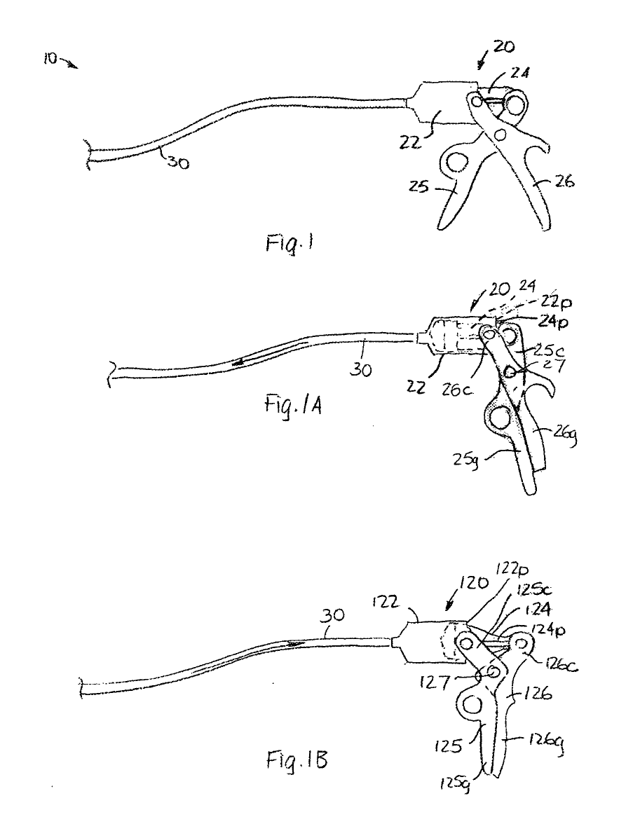

In one aspect, the present invention includes systems for injecting and/or aspirating fluids from the body of a subject. An embodiment of such a system 10 is shown in FIG. 1. Specifically, system 10 includes a syringe 20 and a catheter 30.

Syringe 20 is a hand-held, manually operated syringe. As used herein, the term “hand-held,” when used with the term “syringe,” includes syringes that are configured to be held within one hand of a user (e.g., a physician or other healthcare provider). The phrase “manually operated,” when used in conjunction with the term “syringe,” indicates that the syringe may be hand operated and, when used in tandem with “hand-held” and “syringe,” signifies that the syringe may be held and operated with the same (i.e., one) hand).

In addition to being hand-held and manually operable, syringe 20 provides its user with both a mechanical advantage and tactile feedback. Non-limiting examples of such a syringe include those described in U.S. Pat. No. 7,041,084, in U.S. Patent Application Publication US-2006-0270996-A1, in U.S. patent application Ser. No. 11/431,420, and in U.S. Provisional Patent Application Ser. Nos. 60/853,817 and 60/853,878, the entire disclosure of each of which is hereby incorporated herein by this reference.

Syringe 20 includes a barrel 22, a plunger 24, and a pair of handles 25 and 26 associated with barrel 22 and plunger 24.

In the embodiment of syringe 20 depicted in FIG. 1A, which is an injection syringe, a coupling end 25c of one handle 25 is associated with (e.g., coupled to) a proximal end 24p (relative to a user of syringe 20) of plunger 24, while a coupling end 26c of the other handle 26 is associated with (e.g., coupled to) a proximal end 22p of barrel 22. Handles 25 and 26 are joint at a pivot point 27. When grasping, or bottom, ends 25g and 26g of handles 25 and 26 are forced toward one another (e.g., squeezed together), handles 25 and 26 pivot about pivot point 27 and plunger 24 is forced distally into barrel 22.

In an aspiration syringe 120, shown in FIG. 1B, where handle 125 is bent to resemble a “V.” The point of the “V” of handle 125 crosses, or overlaps, handle 126 at a pivot point 127 where handles 125 and 126 are coupled to each other. A coupling end 125c of handle 125 is associated with a proximal end 122p of barrel 122, while a coupling end 126c of handle 126 is associated with a proximal end 124p of plunger 124. Thus, when grasping, or bottom, ends 125g and 126g of handles 125 and 126 are forced (e.g., squeezed) together, plunger 124 is drawn proximally out of barrel 120.

With continued reference to both FIG. 1A and FIG. 1B, handles 25 and 26, 125 and 126 are pivotally coupled (e.g., by a hinge) to one another at a pivot point 27, 127. The length of the portion (i.e., coupling end 25c, 26c, 125c, 125c) of each handle 25, 26, 125, 126 between pivot point 27, 127 and plunger 24 or barrel 22 is shorter than the length of the portion (i.e., grasping end 25g, 26g, 125g, 126g) of each handle 25, 26, 125, 126 located beneath pivot point 27, 127 (i.e., on the opposite side of pivot point 27, 127 from barrel 22 and plunger 24) and that are to be gripped by a user's hand. Such an arrangement, which resembles pliers, provides a user of syringe 20 with a mechanical and ergonomic advantage. In addition, handle 25, 125 may be shorter than handle 26, 126, which may provide further mechanical advantage. The mechanical advantage provided by the crossing pivotal arrangement of handles 25 and 26, 125 and 126, as well as by their relative lengths, has recently been found to be useful for quickly forcing fluid through (injection or aspiration) a small inner diameter injection/aspiration element 30 that has been coupled to barrel 22 it has also been discovered that the arrangement of handles 25 and 26, 125 and 126 enables a user to maintain a constant rate of injection or aspiration over the entire course of travel of plunger 24 through barrel 22.

In different implementations, syringes 20 with barrels 22 of different volumes may be used. Without limiting the scope of the present invention, syringe 20 may include a barrel having a volume of 10 ml, 12.5 ml, 25 ml, 50 ml, etc. In some embodiments, handles 25 and 26, 125 and 126 may be configured to be reused with different single-use cartridges that include syringe barrels 22, plungers 24, and plunger tips 24t, which may be configured for quick assembly with and disassembly from handles 25 and 26, 125 and 126.

One specific embodiment of injection/aspiration element 30 is a catheter. A catheter 30 that may be used in system 10 may have a length of at least about 65 cm. Catheter 30 may have an outer dimension (e.g., outer diameter, etc.), or size, of about five French (e.g., 1⅔ mm) or less (e.g., four French (1⅓ mm), three French (1 mm), etc.). An inner dimension (e.g., inner diameter) of such a catheter may be about 0.047 inch (about 1.2 mm) or smaller. Alternatively, injection/aspiration element 30 may by a dilation catheter.

System 10 may be used in a variety of procedures, including, but not limited to, angiography and angioplasty procedures. Of course, the specific features of syringe 20 (e.g., injection, aspiration, extent of mechanical advantage, etc.) and catheter 30 (e.g., configured for use with contrast media, balloon, etc.) depend upon the procedure in which system 10 is to be used, as well as the acts that are to be performed by way of the procedure.

Ordinarily, when a catheter 30 of the type, or size, used in system 10 are used with hand-held, manually operated syringes, the rates at which fluids are forced through catheter 30 are undesirably slow for many procedures.

### EXAMPLE 1

The following TABLE presents data obtained using a 10 ml control syringe available from Argon Medical Devices Inc. of Athens, Tex., and a 12 ml control syringe available from Abbott Laboratories or Abbott Park, Ill., with a variety of catheters. Specifically, the catheters that were used included a 2.3 mm TURBO ELITE laser catheter manufactured by the Spectranetics Corporation of Colorado Springs, Colo., which has a length of 120 cm and a lumen dimension (e.g., internal diameter) of 0.020 inch; a 5 F (lumen dimension (e.g., internal diameter) of 0.038 inch to 0.052 inch), 65 cm long COBRA catheter from Merit Medical Systems, Inc., of Murray, Utah; and a 4 F (lumen dimension (e.g., internal diameter) of 0.038 inch to 0.044 inch), 100 cm long JUDKINS catheter, also from Merit Medical Systems. Each syringe-catheter combination was tested five times. The test protocol included inspection of the syringes and catheters for visible defects, filling the syringes to capacity with water (which the International Organization for Standardization (ISO) employs as a standard in testing catheters and syringes and is affected proportionately to contrast) connecting the catheters to the syringes, and determining the amount of time required to expel all of the contents of the syringe into the catheter. The same person performed each test in the order listed in TABLE 1 (i.e., 10 ml control syringe with each catheter listed, left-to-right, then 12 ml control syringe with each catheter listed, left-to-right) using the same hand, with three to four minutes of-rest between tests. The results for each syringe-catheter combination were averaged, as provided in the following TABLE:

TABLE 1

| | | | 4F | | 5F | | |
|---|---|---|---|---|---|---|---|
| | Spectranetics | | 100 cm | | 65 cm | | |
| | 2.3 mm | | Catheter | | Catheter | | |
| | Turbo Full | | Full | 4F | Full | 5F | |
| | Syringe | Spectrantetics | Syringe | 100 cm | Syringe | 65 cm | |
| | Injection | 2.3 mm Turbo | Injection | Catheter | Injection | Catheter | Pass = No |
| Device | Time | ml/sec | Time | Ml/sec | Time | ml/sec | Leaks |
| 10 ml Argon | 6.5 | 1.8 | 3.5 | 3.4 | 2.1 | 5.7 | Yes |
| 12 ml Abbott | 7.1 | 1.8 | 3.6 | 3.3 | 2.3 | 5.2 | Yes |

### EXAMPLE 2

The same procedure was used to test several systems 10 (FIG. 1) that incorporate teachings of the present invention. Specifically, 12.5 ml, 25 ml, and 50 ml syringes of the type described in U.S. Pat. No. 7,041,084, in U.S. Patent Application Publication US-2006-0270996-A1, in U.S. patent application Ser. No. 11/431,420, referred to as “POWRsyringe,” were tested with the same types of catheters as those used in EXAMPLE 1. The following data demonstrate the significant increase in fluid transport rate for the 12.5 ml syringe, as well as the utility in using larger (e.g., 25 ml and 50 ml) syringes to manually drive fluids through catheters that are smaller than 6 French in size.

TABLE 2

| | | | 4F | | 5F | | |
|---|---|---|---|---|---|---|---|
| | Spectranetics | | 100 cm | | 65 cm | | |
| | 2.3 mm | | Catheter | | Catheter | | |
| | Turbo Full | | Full | 4F | Full | 5F | |
| | Syringe | Spectrantetics | Syringe | 100 cm | Syringe | 65 cm | |
| | Injection | 2.3 mm Turbo | Injection | Catheter | Injection | Catheter | Pass = No |
| Device | Time | ml/sec | Time | ml/sec | Time | ml/sec | Leaks |
| 12.5 ml | 4.5 | 2.8 | 2.5 | 5.0 | 1.0 | 12.5 | Yes |
| 25 ml | 11.5 | 2.2 | 6.5 | 3.8 | 3.2 | 7.8 | Yes |
| 50 ml | N/A | N/A | 12.5 | 2.0 | 10.5 | 2.4 | Yes |

These data show that contrast may be delivered in angiography procedures by way of single-hand (manual) operation of a 12.5 ml POWRsyringe in conjunction with 5F and 4F catheters and by way of one-handed operation of a 25 ml POWRsyringe in conjunction with a 5F catheter. Angiography in this manner will minimize discomfort to the subject on whom (which) the procedure is being formed while delivering contrast at a sufficient rate to optimize the density of contrast-enhanced images, to minimize wastage of contrast, and to minimize the potential for nephropathy.

In addition, these data show the feasibility of using catheters with very small (e.g., about 0.020 inch (about 0.5 mm)) inner diameters with hand-held, (single) hand-operated syringes, as well as the feasibility of using relatively large volume hand-held syringes (e.g., syringes with 25 ml barrels, 50 ml barrels, etc.), with catheters that are smaller than 6F in size (e.g., 5F and 4F catheters). For example, a syringe with a barrel that holds 25 ml could be used to force fluid through a 4F catheter at a rate of about 3.0 ml/sec or faster. As another example, a syringe with a barrel that holds 50 ml could be used to introduce contrast at a substantially constant rate of 1 ml/sec, 2 ml/sec, or faster, such as in contrast-enhanced CT or MRI imaging procedures).

It is also apparent that syringes with smaller barrels (e.g., 10 ml, 5 ml, 3 ml, etc.) could be used with a 5F catheter to deliver fluid at rates of 10 ml/sec and faster, with a 4F catheter to deliver fluid at rates of at least 5.0 ml/sec, or with a catheter having an inner diameter of 0.020 inch (about 0.5 mm) at a rate of at least 2.0 ml/sec.

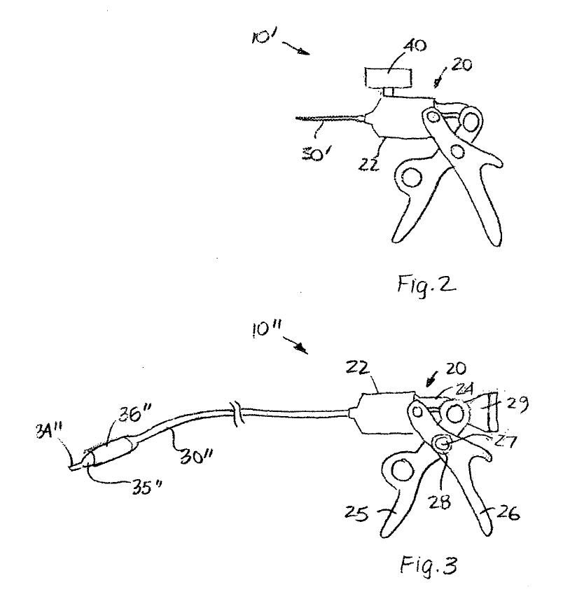

The inner diameters of the lumens of the catheters used in EXAMPLE 2 translate well to the inner diameters of needles. Accordingly, another embodiment of a system 10′ that incorporates teachings of the present invention is shown in FIG. 2. System 10′ also includes a syringe 20 but, in place of or in addition to the catheter 30 of system 10 (FIG. 1), system 10′ includes a needle 30′.

When system 10′ is to be used in procedures where it is useful or necessary to monitor pressure, a pressure gauge 40 of a suitable, known type (e.g., a manometer, etc.) may also be included. Pressure gauge 40 may be in fluid communication with an interior of a barrel 22 of syringe 20.

When system 10′ is used in discography procedures, needle 30′ may be connected directly to barrel 22, or it may be connected to barrel 22 by way of a catheter or other tubing. The size of needle 30′ may be less than 16 gauge in size. As an example, when a 20 gauge needle (i.e., having an outer diameter of about 0.036 inch ((about 0.9 mm) and an inner diameter of about 0.025 inch (about 0.6 mm)) with a length of at least about 5 cm is employed, a relatively small 16 gauge needle (i.e., having an outer diameter of about 0.065 inch (about 1.7 mm) and an inner diameter of about 0.047 inch (about 1.2 mm)) or 17 gauge needle (i.e., having an outer diameter of about 0.058 inch (about 1.5 mm) and an inner diameter of about 0.042 inch (about 1.1 mm)) may be used as the guide needle. When needles that are smaller than 20 gauge in size are used, guide needles that are smaller than 16 gauge may likewise be used.

In the discography process, syringe 20 that includes a barrel 22 with a volume of as small as about 5 ml may be operated with a single hand of a user, including introducing the desired amount of pressure into the intervertebral disk, holding the pressure, and releasing the pressure. Since only one hand is required, the other hand is freed for other purposes, such as holding the needle, catheter, or tubing in place, stabilizing the subject, or the like.

Turning now to FIG. 3, an embodiment of a system 10″ in which a proximal end 32Δ of an angioplasty catheter 30″ is coupled to a barrel 22 of syringe 20.

A pressure gauge 40 may be associated with syringe 20, for example, in fluid communication with an interior of barrel 22 of syringe 20.

A distal end 34″ of angioplasty catheter 30″ is configured for insertion into a blood vessel of the body of a subject. Distal end 34″ includes a balloon 35″, and may include a stent 36″ on balloon 35″.

In an example of the use of system 10″, syringe 20 may be used to inflate balloon 35″. Specifically, bottom ends of handles 25, 26 (i.e., the portions of handles 25 and 26 that are located beneath a pivot point 27) are forced together, forcing a plunger 24 of syringe 20 into barrel 22. As plunger 24 moves further into barrel 22, fluid (e.g., air) within barrel 22 is displaced into catheter 30″ and, eventually, to balloon 35″.

It has only recently been determined that a syringe 20 having a barrel 22 with a displacement volume of about 7 ml may be operated with one hand to generate and hold as much as about 20-24 atmospheres (atm.) of pressure within balloon 35″ without the requirement of locks or microadjustment mechanisms. A 12.5 ml barrel 22 may be used with syringe 20 to generate and hold as much as about eight atmospheres to about 12 atm. of pressure within balloon 35″.

When locks or microadjustment mechanisms are associated with the handles 25, 26 of a syringe 20 according to the present invention, even more pressure may be generated and held. For example, using a syringe 20 that includes locks and microadjustment features (see, e.g., U.S. Provisional Patent Application 60/853,878) and that includes a barrel 22 with a volume of 12.5 ml, up to about 30 atm. of pressure may be generated within an angioplasty balloon 35″. Similarly, it has been discovered that a syringe 20 with a barrel displacement volume of about 25 ml may be used to generate as much as about four atm. to about six atm. in balloon 35″ with one hand, and up to about 30 atm. of pressure within balloon 35″ when locks or microadjustment mechanisms are associated with the handles of the syringe. When barrel 22 of syringe 20 has a displacement volume of about 50 ml, it may be used to generate as much as about two atm. to about three atm. of pressure within balloon 35″ when one hand is used to operate syringe 20, and up to about 15 atm. of pressure when locks or microadjustment mechanisms are associated with the handles of the syringe.

A method of using a hand-held, hand-operated syringe of the type described in U.S. Provisional Patent Application 60/853,878 (fitted with a suitable pressure gauge of known configuration) in an angioplasty procedure includes inflation of balloon 35″ to nominal pressure by operating syringe 20, via handles 25 and 26, with one hand. The user's other hand may be used to hold catheter 30″ in place (and to prevent removal of catheter 30″ from the body of a subject during the inflation process). Once nominal pressure has been obtained, the same hand that has been used to move handles 25 and 26 may be used to operate a locking element 28 that retains a desired location between handles 25 and 26. Additionally, that same hand may be used to operate a microadjustment mechanism 29 associated with handles 25 and 26 to finely adjust the relative positions of handles 25 and 26, as well as the position of plunger 24 within barrel 22, and, thus, further increase or decrease (by small amounts) pressure within balloon 35″ (which pressure may be visually displayed by pressure gauge 40 (FIG. 2)), if necessary. By releasing locking element 28 (e.g., with the same hand that grasps handles 25 and 26) and allowing the bottom ends of handles 25 and 26 to separate from one another as the resilience of balloon 35″ and of the vessel within which balloon 35″ (and, possibly, of surrounding tissues) is disposed forces fluid out of balloon 35″, proximally through catheter 30″, and back into barrel 22 of syringe 20.

In other embodiments, a system that includes syringe 20 may be used for a variety of other purposes (e.g., injection of orthopedic glue, hemodynamic monitoring (e.g., invasive cardiac output measurement), injection or aspiration of viscous substances through elements, such as catheters, needles, or the like, with inner diameters of 0.052 inch or smaller, etc.) in place of more expensive apparatus.

The simple design and manufacture of syringe 20 makes it much less expensive than conventional angioplasty inflators, power syringes, discography syringes, and similar devices. It may, therefore, be considered to be a disposable (single-procedure use) device, which eliminates the need for sterilization and reduces contamination issues that may be presented due to incomplete or ineffective sterilization. Costs may be further reduced in embodiments where handles 25 and 26 are configured to be reused with different, disposable syringe barrels 22, plungers 24, and plunger tips, which may configured as single-use cartridges that are configured for quick assembly with and disassembly from handles 25 and 26. In embodiments where handles 25 and 26 are reused, less waste is created.

Although the foregoing description contains many specifics, these should not be construed as limiting the scope of the present invention, but merely as providing illustrations of some exemplary embodiments. Similarly, other embodiments of the invention may be devised which do not depart from the spirit or scope of the present invention. Features from different embodiments may be employed in combination. The scope of the invention is, therefore, indicated and limited only by the appended claims and their legal equivalents, rather than by the foregoing description. All additions, deletions, and modifications to the invention, as disclosed herein, which fall within the meaning and scope of the claims are to be embraced thereby.

What is claimed:

1. An injection system, comprising:
a syringe, including:
a barrel;
a plunger; and
a pair of handles intersecting and pivotally connected to each other at somewhat central, intersecting location, a first of the handles pivotally associated with the barrel, a second of the handles pivotally associated with the plunger, ends of the handles configured to be grasped and operated by a single hand of a user; the second of the handles is shorter than the first of the handles, and
a catheter including a proximal end coupled to and in fluid communication with the barrel of the syringe and a distal end for insertion into a body of a subject, the catheter having a length of at least about 65 cm, an outer diameter of less than 6 French and a primary lumen with an inner diameter of less than 0.050 inch.

2. The injection system of claim 1, wherein the syringe imparts the user with a mechanical advantage sufficient to force water from the barrel, through the catheter, and out of the distal end of the catheter at a rate of at least 5 ml per second when the user manually operates the pair of handles with the single hand.

3. The injection system of claim 2, wherein the barrel of the syringe has a volume of about 10 ml , the catheter is a 5 French catheter, and the syringe imparts the user with a mechanical advantage sufficient to force water from the barrel, through the catheter, and out of the distal end of the catheter at a rate of at least 10 ml per second when the user manually operates the pair of handles with the single hand.

4. The injection system of claim 2, wherein the barrel of the syringe has a volume of about 10 ml, the catheter is a 4 French catheter, and the syringe imparts the user with a mechanical advantage sufficient to force water from the barrel, through the catheter, and out of the distal end of the catheter at a rate of at least 5 ml per second when the user manually operates the pair of handles with the single hand.

5. The injection system of claim 2, wherein the barrel of the syringe has a volume of about 25 ml, the catheter is a 5 French catheter, and the syringe imparts the user with a mechanical advantage sufficient to force water from the barrel, through the catheter, and out of the distal end of the catheter at a rate of at least 5 ml per second when the user manually operates the pair of handles with the single hand.

6. The injection system of claim 2, wherein the barrel of the syringe has a volume of about 25 ml, the catheter is a 4 French catheter, and the syringe imparts the user with a mechanical advantage sufficient to force water from the barrel, through the catheter, and out of the distal end of the catheter at a rate of at least 3.0 ml per second when the user manually operates the pair of handles with the single hand.

7. The injection system of claim 2, wherein the barrel of the syringe has a volume of at least about 10 ml ml, the catheter has an inner diameter of about 0.020 inch, and the syringe imparts the user with a mechanical advantage sufficient to force water from the barrel, through the catheter, and out of the distal end of the catheter at a rate of at least 2.0 ml per second when the user manually operates the pair of handles with the single hand.

8. An angiography method, comprising:
introducing contrast media into a barrel of a manually operable syringe with crossing handles configured to be held and operated with one hand;
securing a proximal end of a catheter having an outer diameter of less than 6 French to a barrel of a manually operable syringe;
inserting a distal end of the catheter into a location of a body of a subject to be externally visualized; and
manually forcing a plunger of the syringe into the barrel of the hand-held syringe by squeezing the crossing handles with one hand so as to cause the contrast media to exit the distal end of the catheter at a rate of at least about 10 ml per second.

9. The angiography method of claim 8, further comprising:
externally visualizing the contrast media.

10. A method for reducing contrast media wastage in angiography, comprising:
introducing contrast media into a barrel of a manually operable syringe configured to be held within and operated by a single hand of a user;
securing a proximal end of a catheter to the barrel;
inserting a distal end of the catheter into a location of a body of a subject to be externally visualized; and
manually forcing a plunger of the syringe into the barrel by squeezing crossing handles of the syringe with one hand to force the contrast media through the catheter at a substantially constant rate of at least 5 ml/sec.

11. The method of claim 10, wherein, during squeezing, a user is provided with tactile feedback.

12. The method of claim 10, wherein introducing comprises introducing the contrast media into a barrel having a volume of at least about 10 ml.

13. The method of claim 10, wherein securing comprises securing a proximal end of a catheter having an inner diameter of about 0.052 inch or less to the barrel.

14. The method of claim 10, wherein introducing comprises introducing the contrast media into a barrel having a volume of at least about 25 ml.

15. The method of claim 14, wherein securing comprises securing a catheter having an outer diameter of about 6 French or less and a length of about 65 cm or more to the barrel.

16. A discography method, comprising:
introducing media into a barrel of a manually operable syringe including a pair of crossed handles that is configured to be held within and operated by a single hand of a user;
coupling a discography needle directly or indirectly to a distal end of the barrel;
introducing the discography needle into a nucleus of an intervertebral disk of a subject; and
squeezing the handles to force the media into the nucleus.

17. The discography method of claim 16, further comprising:
visualizing the media within the intervertebral disk.

18. The discography method of claim 16, further comprising:
monitoring a pressure applied to the media with the syringe.

19. The discography method of claim 16, further comprising:
holding the needle in place with the user's other hand, during the act of squeezing.

20. An inflation method employing a catheter and a syringe, comprising:
introducing a distal end of at least one of a balloon catheter and a dilation catheter to a desired site within a body of a subject;
coupling a proximal end of the at least one of the balloon catheter and the dilation catheter to a distal end of a barrel of a syringe;
grasping crossing handles of the syringe with a single hand; and
squeezing grasping ends of the crossing handles together with the single hand to force fluid from the barrel and into the catheter to expand at least one of a balloon of the balloon catheter and the dilation catheter.

21. The inflation method of claim 20, wherein squeezing further includes holding the crossed handles together to hold the balloon or the dilation catheter in an expanded state.

22. The inflation method of claim 20, further comprising:
holding the at least one of the balloon catheter and the dilation catheter in place with the other hand of the user during the act of squeezing.

23. The inflation method of claim 20, further comprising:
monitoring a pressure within the at least one of the balloon catheter and the dilation catheter.

24. The inflation method of claim 20, further comprising:
releasing the crossing handles to enable movement of grasping ends of the crossing handles away from one another and to release pressure within and deflate the at least one of the balloon catheter and the dilation catheter.
